(19) United States
(12) Patent Application Publication
(10) Pub. No.: US 2006/0062864 A1
(43) Pub. Date: Mar. 23, 2006

(54) Weight loss composition and method

(76) Inventor: Edward L. McCleary, Incline Village, NV (US)

Correspondence Address: PATTON BOGGS, 1660 LINCOLN ST, SUITE 2050, DENVER, CO, 80264, US

(21) Appl. No.: 11/271,350

(22) Filed: Nov. 12, 2005

Related U.S. Application Data
(63) Continuation-in-part of application No. 11/111,542, filed on Apr. 21, 2005.
Application No. 11/111,542 is a continuation-in-part of application No. 10/986,924, filed on Nov. 12, 2004.
(63) Continuation-in-part of application No. 10/462,958, filed on Jun. 17, 2003.
Application No. 10/462,958 is a continuation-in-part of application No. 09/749,584, filed on Dec. 28, 2000, now Pat. No. 6,579,866.
(63) Continuation-in-part of application No. 10/890,067, filed on Jul. 12, 2004.
Application No. 10/890,067 is a continuation-in-part of application No. 10/462,958, filed on Jun. 17, 2003.
Application No. 10/890,067 is a continuation-in-part of application No. 10/616,674, filed on Jul. 10, 2003.
(60) Provisional application No. 60/520,466, filed on Nov. 14, 2003.
(60) Provisional application No. 60/536,286, filed on Jan. 13, 2004.

(51) Int. Cl. A61K 36/87; A61K 31/7048; A61K 31/198; A61K 36/82

(52) U.S. Cl. 424/766; 514/184; 514/27; 514/456; 514/564; 514/548; 424/729; 424/769

(57) ABSTRACT

Improved compositions and methods are useful in the treatment and prevention of impulsive, compulsive, reward-seeking, and other addictive behaviors, including applications involving the behavioral/cognitive process of assigning importance, maintaining attention, and assessing salience. The composition includes Vitex agnus-castus (Chastetree berry) extract in an effective amount for supporting and promoting a healthy lifestyle free from addictive behavior or for preventing or treating addictive behavior. The composition also includes garcinia cambogia, aspartic acid, pyruvate, EPA (eicosapentanoic acid), biotin, L-carnitine, and chromium polynicotinate.

### CROSS REFERENCE TO RELATED APPLICATIONS

This application is a continuation-in-part of U.S. patent application Ser. No. 11/111,542 filed on Apr. 21, 2005, which itself is a continuation-in-part of U.S. patent application Ser. No. 10/986,924 filed Nov. 12, 2004. This application is also a continuation-in-part of U.S. patent application Ser. No. 10/462,958 filed on Jun. 17, 2003, in the name of Edward Larry McCleary, which itself is a continuation-in-part of U.S. patent application Ser. No. 09/749,584, filed Dec. 28, 2000, in the name of Edward Larry McCleary, now U.S. Pat. No. 6,579,866, which issued Jun. 17, 2003. This application is also a continuation-in-part of U.S. patent application Ser. No. 10/890,067 filed on Jul. 12, 2004, which itself is a continuation-in-part of U.S. patent application Ser. No. 10/462,958 filed on Jun. 17, 2003, and is also a continuation-in-part of U.S. patent application Ser. No. 10/616,674 filed Jul. 10, 2003. Through the above-mentioned U.S. patent application Ser. No. 10/890,067, this application also claims the benefit of U.S. Provisional Patent Application No. 60/520,466 filed Nov. 14, 2003 and U.S. Provisional Patent Application Ser. No. 60/536,286 filed Jan. 13, 2004. All of the above-cited patent applications and patent are incorporated in this disclosure to the same extent as though fully disclosed herein.

### FIELD OF THE INVENTION

This invention relates in general to nutraceuticals, including functional foods, dietary supplements, nutritional supplements, medical foods, botanical drugs, and drugs. In particular, it relates to nutraceuticals and methods that are useful for promoting weight loss and weight maintenance.

### BACKGROUND OF THE INVENTION

Approximately 30% to 50% of the population of westernized societies is overweight. Obesity is a serious health problem in the United States and many other countries. About 90 million citizens are now considered obese. Obesity is known to be associated with many serious diseases and metabolic disorders including small, dense LDL syndrome, glucolipoxia, premature aging, memory loss, endothelial dysfunction, vascular disease, hypertension, postprandial hyperlipidemia, insulin resistance, hyperinsulinemia, Syndrome X, hypertriglyceridemia and/or low HDL syndrome, high RQ (respiratory quotient) syndrome, chronic fatigue syndrome, as well as certain types of cancer. In addition, it has psychological implications.

Because of the recognized problem of obesity, many food supplements and medications have been developed to address the problem. Nearly every avenue of weight loss has been explored with these supplements and medications. Appetite suppressants attack the problem by preventing overeating. Stimulants address the problem by causing the user to burn more energy. More recently, fat and/or carbohydrate blockers have become available which prevent the digestion and/or absorption of fat and/or carbohydrates in the intestines. A more sophisticated approach utilizes thermogenic agents to alter the metabolism to cause the body to burn more energy. A related relatively sophisticated approach starts from the premise that the metabolic functions of our bodies are not well adapted to current dietary and lifestyle choices. That is, the human body that was developed over a period when humans had to expend large amounts of energy to gather or hunt for food, and lived in unheated buildings requiring further expenditures of energy to stay warm, may be maladapted for a sealed, controlled environment and a lifestyle that requires sitting before a computer for long periods and provides inexpensive fast foods. However, for many people, none of these weight loss and weight maintenance solutions work. Since obesity has such huge economic and human consequences, it would be highly desirable to have a weight loss composition and formula that is especially effective for persons who have the most problem keeping weight maintained.

### BRIEF SUMMARY OF THE INVENTION

The invention solves the above and other related problems by the realization that obesity has aspects of addiction. The invention provides a weight loss composition and method that addresses the impulsive eating or bingeing aspect of obesity, as well as other addictive behaviors. The invention provides new therapeutic, preventive, and weight maintenance strategies that focus upon both direct and indirect modulation of dopaminergic function and restoration of associated brain circuitry. The invention provides a weight loss composition and method that modulates the level of brain dopamine (DA), and particularly dopamine type D2 (DA D2) receptors.

The invention provides a composition for promoting weight loss or weight maintenance, the composition comprising a Vitex agnus-castus (Chastetree berry or Chasteberry) extract in an effective amount for promoting weight loss or weight maintenance. Preferably, the composition further includes: an ingredient selected from the group consisting of hydroxycitric acid and garcinia cambogia; an ingredient selected from the group consisting of aspartic acid, aspartate, and pyruvate; and EPA (eicosapentanoic acid), preferably in the form of fish oil, and most preferably, dessicated fish oil. Preferably, the composition further includes biotin, L-carnitine, and chromium, preferably in the form of chromium polynicotinate. In the preferred formulation, the composition includes Vitex agnus-castus (Chastetree berry or Chasteberry) extract; an ingredient selected from the group consisting of hydroxycitric acid and garcinia cambogia; an ingredient selected from the group consisting of aspartic acid, aspartate, and pyruvate; EPA; biotin; L-carnitine; and chromium. Most preferably, the composition includes Vitex agnus-castus, Garcinia camogia, aspartic acid, pyruvate, EPA, biotin, L-carnitine, and chromium polynicotinate.

The composition may also include medium chain triglycerides, green tea leaf extract containing EGCG (epigallocatechingallate), and 5-HTP (5-hydroxytryptophan).

The invention also provides a composition for supporting and promoting a healthy lifestyle free from addictive behavior or for preventing or treating addictive behavior, the composition comprising a Vitex agnus-castus (Chastetree berry or Chasteberry) extract in an effective amount for supporting and promoting a healthy lifestyle free from addictive behavior or for preventing or treating addictive behavior. Preferably, the composition further comprises huperzine in an effective amount for supporting and promoting a healthy lifestyle free from addictive behavior or for preventing or treating addictive behavior. Preferably, the composition further includes biotin, L-carnitine, chromium polynicotinate, aspartic acid, and garcinia cambogia. Preferably, the composition may also include an ingredient selected from the group consisting of: EPA (eicosapentanoic acid), medium chain triglycerides, green tea leaf extract containing EGCG (epigallocatechingallate), and 5-HTP (5-hydroxytryptophan).

In an alternative embodiment, the composition includes Vitex agnus-castus extract and huperzine and further includes an ingredient selected from the group consisting of DMAE (dimethylaminoethanol) and choline.

In another embodiment, the composition comprises Vitex agnus-castus extract and huperzine, and also includes vitamin B1, vitamin B2, vitamin B3, vitamin B6, ferrous sulfate, magnesium, choline, DHA (docosahexanoic acid), and lipoic acid. Additionally, the composition may include taurine.

In another alternative embodiment, the composition of the invention includes Vitex agnus-castus and 5-HTP (5-hydroxytryptophan). Preferably, the composition also includes tyrosine.

In any of the above embodiments, the composition may be included in a delivery vehicle comprising a vehicle selected from the group consisting of an edible film; a breath-care strip, mint, or lozenge; a food; a beverage; a spice; a condiment; and a salad dressing.

The compositions above preferably also include an ingredient selected from the group consisting of glutamate agonists, antagonists, and precursors; GABA agonists, antagonists, and precursors; serotonin agonists, antagonists, and precursors; and agents that affect the levels and interactions of the forgoing ingredients, the ingredient provided in an effective amount for supporting and promoting a healthy lifestyle free from addictive behavior or for preventing or treating addictive behavior. The compositions above preferably also include ingredients to address nutritional deficiencies due to the addictive behavior, or ingredients to replenish or restore nutrients depleted by drugs taken for the addictive behavior. The compositions above preferably further include ingredients to replenish nutrients depleted during the withdrawal of any reinforcer that is the object of the addictive behavior.

The invention also provides a method for supporting and promoting a healthy lifestyle free from addictive behavior or for preventing or treating addictive behavior in a human being, the method comprising orally or parenterally administering to the human being, for an effective period, a composition comprising a Vitex agnus-castus extract in an effective amount for supporting and promoting a healthy lifestyle free from addictive behavior or for preventing or treating addictive behavior in a human being. Preferably, the method further includes administering an ingredient selected from the group consisting of huperzine and 5-HTP (5-hydroxytryptophan), the ingredient provided in an effective amount for supporting and promoting a healthy lifestyle free from addictive behavior or for preventing or treating addictive behavior. Preferably, the method further includes administering an ingredient selected from the group consisting of glutamate agonists, antagonists, and precursors; GABA agonists, antagonists, and precursors; serotonin agonists, antagonists, and precursors; and agents that affect the levels and interactions of the forgoing ingredients, the ingredient provided in an effective amount for supporting and promoting a healthy lifestyle free from addictive behavior or for preventing or treating addictive behavior. Preferably, the method further comprises administering an ingredient selected from the group consisting of: ingredients to address nutritional deficiencies due to the addictive behavior; ingredients to replenish or restore nutrients depleted by drugs taken for the addictive behavior; and ingredients to replenish nutrients depleted during the withdrawal of any reinforcer that is the object of the addictive behavior.

The invention not only provides a composition for supporting and promoting a healthy lifestyle free from addictive behavior or for preventing or treating addictive behavior in a human being, in particular a composition for promoting weight loss and weight maintenance, it also provides methods of delivering the composition that assists in its daily use. Numerous other advantages and features of the invention will become apparent from the following detailed description.

### DETAILED DESCRIPTION OF THE INVENTION

In this disclosure, the term addictive behavior includes behavior commonly categorized under the term addiction, as well as impulsive, compulsive, craving, and reward-seeking behavior, and including behaviors involving the behavioral/cognitive process of assigning importance, maintaining attention, and assessing salience.

The invention recognizes that the characteristics of compulsive overeating are consistent with at least some of the characteristics of drug-using behavior. Both compulsive overeating and drug addiction manifest the inability to refrain from using a reinforcer and its compulsive administration. Thus, DA D2 decrements are unlikely to be specific for any one of these compulsive behavioral disorders, including obesity, and may relate to vulnerability for addictive disorders in general. Obese individuals have significantly lower DA D2 receptor levels, as do drug-addicted subjects. Lower DA D2 receptor levels in obese individuals would make them less sensitive to reward stimuli, which would make them more vulnerable to food intake as a means to temporarily compensate for this deficit.

Increases in DA secondary to phasic DA cell firing plays an important role in coding rewards and reward-associated stimuli, yet do not code exclusively for reward but also for saliency, which in addition to reward includes aversive, novel, and unexpected stimuli. It is proposed here that DA encodes for the motivation to procure the reward in addition to encoding for the reward itself This view about the role of DA in reinforcement provides a different perspective about drugs of abuse, indicating that drugs are reinforcing not just because they are pleasurable but also because by increasing DA they are being processed as salient stimuli that will inherently motivate further drug procurement.

Just as compulsive behavior, such as drug seeking, is encoded by DA (which attaches significance to such stimuli), hypodopaminergic conditions such as various attentional disorders may be unable to attach appropriate salience. It may be, then, that all stimuli are viewed as being equi-salient. This may make it impossible to prioritize, and may provide insight into the inability to focus or choose something specific to focus upon. From this perspective, it is not difficult to imagine a relationship between salience and inattention or impulsiveness. The converse may apply to the compulsive behavior of a drug addict wherein specific behavior (i.e., drug taking) is assigned salience, and is thereby reinforcing.

PET scans have shown that the reinforcing effects of drugs of abuse are contingent upon large and rapid increases in extra-cellular DA. In addition, similar studies have also documented a role for DA in motivation, which appears to be encoded by both fast, as well as less dramatic, DA increases. Since DA neurons fire in response to salient stimuli, supra-physiological activation by drugs is likely to be experienced as highly salient, in turn driving attention, arousal-conditioned learning, and motivation. Imaging studies have shown in drug addicts that DA function is markedly disrupted, i.e., decreases in DA release and in DA D2 receptors occur in striatal regions, and this is associated with decreased activity of the orbito-frontal cortex (the neuroanatomical region involved with salience attribution and motivation, and implicated in compulsive behaviors) and the cingulate gyrus (the neuroanatomical region involved with inhibitory control and attention/focus/vigilance and which is implicated in impulsiveness). It is postulated here that decreased DA function results in decreased sensitivity to non drug-related stimuli and disruption of frontal inhibition. Such abnormalities have been described in persons afflicted with attention deficit disorder (ADD), attention deficit hyperactivity disorder (ADHD), and other conditions with altered attention processing. It is, therefore, no surprise that this population suffers from excessive addictive behavior and that appropriate medical therapy diminishes this risk.

The invention provides new therapeutic, preventive, and health maintenance strategies which focus upon both direct and indirect modulation of dopaminergic function and restoration of associated brain circuitry. This is consistent with the observations that alcohol, cocaine, heroin, marijuana, nicotine use, glucose-bingeing, pathological gambling, excessive video game use, and sex addiction all cause activation and release of brain DA. These aberrant behaviors, each related to self-fulfillment of a hypodopaminergic trait, may all be combined under the rubric of a reward deficiency syndrome. Extracts from the Chastetree berry (Vitex agnus-castus) are germane in this context. They contain a number of compounds or constituents with dopaminergic properties. Specifically, they bind to recombinant DA D2 receptor protein with subsequent receptor activation. The chemical identity of the active compounds involves the isolation of a number of diterpenes. Hence, extracts of the Chastetree berry (Vitex agnus-castus) manifest dopaminergic activity and are specifically active at the DA D2 receptor. Herein, the term Vitex agnus-castus extract means any active compound derived from the Vitex agnus-castus plant, which is also referred to as Chastetree, including any compound derived from the Chastetree berry, which may also be referred to as the Chasteberry, and also including any drug or drug ingredient synthesized from the Vitex agnus-castus plant. Based upon the prior discussion, along with the ability of extracts of Vitex agnus-castus to cross the blood-brain barrier, it is seen that there exists clinical applications in a number of addictive conditions including, but not limited to, overeating, overweight, obesity (and related conditions), sugar and chocolate craving, smoking, excessive alcohol consumption, nicotine addiction, narcotic addiction, cocaine, and any number of other addictive drugs, compulsive gambling, and pathological sex addiction. These agents directly augment DA D2 neurotransmission. In so doing, they provide the DA “fix” the body would otherwise be seeking. This mechanism acts to circumvent the reward deficiency and forms the basis for the current therapeutic approach. They will also be effective in conditions manifesting impulsiveness, inattention, difficulty assigning salience, and the like. These extracts may also be combined with additional agents and ingredients having benefit in specific situations; for example, ingredients designed to replenish and restore nutrients or nutritional deficiencies in such addictive individuals, and nutrients depleted during the withdrawal of any drug, nicotine, or other reinforcer that is the object of the addiction.

The compositions according to the invention may be included in foods, “functional foods,” dietary supplements, medical foods, botanical drugs, homeopathic remedies, over-the-counter drugs, prescription drugs, and compounded drugs. They may be useful not only for the treatment of various addictions, but also in the prevention of such behaviors. These extracts and combinations may also be used as appetite suppressants for merely overweight (not obese) individuals, and to restore nutrients depleted in addictive-prone and compulsive individuals, and in those in withdrawal.

Formulations

Each formulation is designed to be taken by human beings, preferably on a daily basis, preferably several times per day for at least two weeks, and then thereafter for as long as is required. Each formulation includes Vitex agnus-castus, preferably as an extract, administered in capsules, soft gel tablets, liquids, etc., and may include other herbal, botanical, or dietary ingredients of the sort used in and permitted in dietary supplements, and any drug or drug ingredient synthesized from Vitex agnus-castus. Administration via the oral, sublingual, subcutaneous, intramuscular, transdermal, or IV route is anticipated. Inclusion in foods, drinks, wafers, chewable formulations, gum, candy, bars, powdered shakes, meal replacements, dietary supplements, etc., are anticipated, as well as inclusion in functional foods, medical foods, drugs, etc. The compositions according to the invention may be included with many other combinations of compounds which are beneficial in the conditions listed, including, but not limited to, vitamins, minerals, amino acids, and other nutrients and dietary ingredients. They may be included with many other combinations of compounds which are beneficial in the conditions listed for use in compounded drugs, prepared by a compounding pharmacist for a specific individual. One skilled in the field, such as a physician, may use medical insight, therapeutic skill, prior knowledge, data from the literature, and so forth to compose the specific formulation for a specific individual.

There is extensive interaction among numerous neurotransmitters in many ways. For example, acetylcholine may modulate dopamine neurotransmission. Therefore, agents that modify cholinergic neurotransmission such as huperzine may be expected to indirectly impact dopaminergic pathways and thereby influence the net effect that administration of Vitex agnus-castus has. This is also true for glutamate agonists, antagonists, and precursors; GABA agonists, antagonists, and precursors; serotonin agonists, antagonists, and precursors; and agents that affect their levels and interactions, and so forth, for most of the known neurotransmitters. One skilled in the art will have the benefit of the medical literature and past training for precise guidance in composing specific formulations based upon these principles.

### EXAMPLE 1

Overweight is a condition associated with the ingestion of a relative excess of calories. Frequently, under such circumstances, food is consumed for reasons other than metabolic need. Under these conditions, it may play a rewarding role and be associated with DA release. The provision of an extract of Vitex agnus-castus alone, or in combination with other agents, may substitute for the foodstuffs as a reinforcer and would act to diminish the reliance upon food, thereby facilitating subsequent weight loss.

Formulation 1:

Vitex agnus-castus capsules: (standardized to casticin)-200 mg taken orally twice per day

Formulation 2:

As above, but including the following per dose or serving size:

| | | |
|---|---|---|
| Biotin | 600 | mcg |
| L-carnitine | 10 | mg |
| Pyruvate | 2000 | mg |
| Aspartic acid | 1500 | mg |
| Garcinia cambogia | 500 | mg |
| EPA (eicosapentanoic acid) | 1 | mg per dose |
| | | or serving size |

One may substitute 250 milligrams of hydroxycitric acid for the Garcinia camobogia, or use smaller amounts of both. One may reduce or delete either the pyruvate of aspartic acid and include respectively larger amounts of the other. Aspartate may be used in place of the aspartic acid. The EPA is preferably provided in the form of fish oil, and preferably dessicated fish oil.

Formulation 3:

As in Formulation 2 above, but including the following per dose or serving size:

| | |
|---|---|
| Chromium polynicotinate | 400 mcg |

Formulation 4:

As in formulation 1 above, but including the following per dose or serving size:

| | | |
|---|---|---|
| Biotin | 600 | mcg |
| L-carnitine | 10 | mg |
| Chromium polynicotinate | 400 | mcg |
| Aspartic acid | 4 | g |
| Garcinia cambogia | 500 | mg |

Formulation 5:

As in Formulation 4 above, but including the following per dose or serving size:

| | |
|---|---|
| EPA (eicosapentanoic acid) | 10 mg per dose or serving size |

Formulation 6:

| | |
|---|---|
| As in Formulations 2, 3, 4, or 5 above, | 5 g per dose or serving size |
| but including Medium Chain Triglycerides | |

Formulation 7:

As in Formulations 2, 3, 4, 5, or 6 above, but including the following per dose or serving size:

Green tea leaf extract containing 100 mg of EGCG (epigallocatechingallate)

Formulation 8:

As in Formulations 2, 3, 4, 5, 6, or 7 above, but including the following per dose or serving size:

| | |
|---|---|
| 5-HTP (5-hydroxytryptophan) | 250 mg |

### EXAMPLE 2

For smoking cessation, one dose or serving size two or three times per day.

Formulation 1:

| | |
|---|---|
| Each dose or serving size consisting of | 50 mg |
| Vitex agnus-castus (standardized to | |
| casticin) | |

Formulation 2:

As in Formulation 1 above, but including per dose or serving size:

| | |
|---|---|
| Huperzine | 50 mcg |

Formulation 3:

As in Formulation 2 above, but including per dose or serving size:

| | |
|---|---|
| DMAE (dimethylaminoethanol) | 150 mg |

Formulation 4:

As in Formulations 2 or 3 above, but including per dose or serving size:

| | |
|---|---|
| Choline | 200 mg |

### EXAMPLE 3

For use in conditions of attentional deficit, impulsiveness, or hyperactivity.

Formulation 1:

| | |
|---|---|
| Vitex agnus-castus | 100 mg per dose or serving |
| (standardized to casticin) | size to be taken two or three times per day. |

Formulation 2:

As in Formulation 1 above, but including per dose or serving size:

| | |
|---|---|
| Huperzine | 100 mcg |

Formulation 3:

As in Formulation 2 above, but including per dose or serving size:

| | |
|---|---|
| Vitamin B1 | 10 mg |
| Vitamin B2 | 20 mg |
| Vitamin B3 | 25 mg |
| Vitamin B6 | 10 mg |
| Ferrous sulfate | 5 mg |
| Magnesium | 150 mg |
| Choline | 50 mg |
| DHA (docosahexanoic acid) | 40 mg |
| Lipoic acid | 30 mg |

Formulation 4:

As in Formulations 2 or 3 above, but also including per dose or serving size:

| | |
|---|---|
| Taurine | 50 mg |

### EXAMPLE 4

To help counteract excessive sweet cravings and as an appetite suppressant generally, taken several times per day and as needed, per dose or serving size:

Formulation 1:

| | |
|---|---|
| Extract of Vitex agnus-castus | 40 mg |
| (standardized to casticin) | |

Formulation 2”

As in Formulation 1 above, plus per dose or serving size:

| | |
|---|---|
| 5-HTP (5-hydroxytryptophan) | 250 mg |

Formulation 3:

As in Formulation 2 above, plus the following per dose or serving size:

| | |
|---|---|
| Tyrosine | 500 mg |

As discussed above, the above compositions according to the invention are preferably administered via the oral, sublingual, subcutaneous, intra-muscular, transdermal, or IV route. Sometimes, such “formal” administration may not be effective, in that taking a dose of a “medicine” each day is difficult enough for healthy people, much less addicts. Therefore, the invention contemplates inclusion of any of the above compositions in foods, drinks, wafers, chewable formulations, gum, candy, bars, powdered shakes, meal replacements, dietary supplements, etc., as well as inclusion in functional foods, medical foods, drugs, etc. For example, the delivery vehicle may comprise an edible film; a breath-care strip, mint, or lozenge; a food, water, beverage, spice, or other food additive; condiment; salad dressing; or other functional food. The preferred foods are: energy bars, salad dressings, condiments (such as steak sauce, mustard, catsup, and soy sauce), vegetable oils, fruit products (such as jellies, jams, and syrups), cereals, trail mix, cookies, pasta, flours (including wheat, soy, oat, and potato flour), whey, chocolate, yogurt, tofu, bagels, baked goods, vegetables, soups, nutritional bars, snacks, crackers, meats, meat products such as lunch meats, and milk products such as ice cream, cheese, and butter. The term “beverage” is used in its common meaning, which does not include water or medicines. The preferred beverages are sports drinks, soft drinks, alcoholic beverages, tea, coffee, milk, and fruit juices.

There have been described novel compositions and methods for promoting compulsion-free health, which can particularly address weight loss in persons who have difficulty losing weight. It should be understood that the specific formulations and methods described herein are exemplary and should not be construed to limit the invention, which will be described in the claims below. For each ingredient specified, it is not necessary to use the amount specified, but rather a wide range of amounts of the ingredient may be used. Further, it is evident that those skilled in the art may now make numerous uses and modifications of the specific embodiments described without departing from the inventive concepts. For example, the appropriate assignment of salience, and/or importance, to various stimuli or tasks is also a part of non-compulsive behavior. The ability to attend and focus, especially for prolonged periods, also falls under the purview of this invention. Included are behaviors of compulsive and/or excessive food consumption; drug taking and/or addiction; smoking; compulsive, excessive, or inappropriate gambling; compulsive or uncontrollable sexual behavior; chocolate or sweet craving; impulsive behavior; difficulty with concentration, attention, and/or focus; and the like. Also contemplated are compositions and methods for dietary supplements to restore nutritive balance, and nutrients lost or depleted during any protocol to address or “break” an addiction. Consequently, the invention is to be construed as embracing each and every novel feature and novel combination of features present in and/or possessed by the compositions and methods described and by their equivalents.

What is claimed as new and desired to be protected by Letters Patent of the United States is:

1. A composition for promoting weight loss or weight maintenance, the composition comprising a Vitex agnus-castus (Chastetree berry or Chasteberry) extract in an effective amount for promoting weight loss or weight maintenance.

2. The composition as in claim 1 and further including: an ingredient selected from the group consisting of hydroxycitric acid and garcinia cambogia; an ingredient selected from the group consisting of aspartic acid, aspartate, and pyruvate; EPA (eicosapentanoic acid); biotin, L-carnitine, and chromium.

3. The composition as in claim 2 wherein said chromium comprises chromium polynicotinate.

4. The composition as in claim 2 wherein said EPA comprises fish oil.

5. The composition as in claim 4 wherein said fish oil comprises dessicated fish oil.

6. The composition as in claim 2, and further comprising an ingredient selected from the group consisting of: medium chain triglycerides, green tea leaf extract containing EGCG (epigallocatechingallate), and 5-HTP (5-hydroxytryptophan).

7. A composition as in claim 1, said composition included in a delivery vehicle selected from the group consisting of an edible film; a breath-care strip, mint, or lozenge; a food; a beverage; a spice or other food additive; a condiment; and a salad dressing.

8. A method for promoting weight loss or weight maintenance in a human being, said method comprising orally or parenterally administering to the human being, for an effective period, a composition comprising a Vitex agnus-castus extract in an effective amount for promoting weight loss or weight maintenance in a human being.

9. A method as in claim 8 wherein said method further includes administering a composition further including: garcinia cambogia, aspartic acid, pyruvate, EPA (eicosapentanoic acid), biotin, L-carnitine, and chromium in effective amounts for promoting weight loss or weight maintenance.

10. A method as in claim 8 wherein said method further includes administering a composition further including an ingredient selected from the group consisting of: medium chain triglycerides, green tea leaf extract containing EGCG (epigallocatechingallate), and 5-HTP (5-hydroxytryptophan).

11. A method as in claim 8 wherein said administering comprises placing said composition in a delivery vehicle selected from the group consisting of an edible film; a breath-care strip, mint, or lozenge; a food; a beverage; a spice or other food additive; a condiment; and a salad dressing.
